(19) United States
(12) Patent Application Publication
(10) Pub. No.: US 2022/0022728 A1
(43) Pub. Date: Jan. 27, 2022

(54) MEDICAL SYSTEM, INFORMATION PROCESSING DEVICE, AND INFORMATION PROCESSING METHOD

(71) Applicant: SONY GROUP CORPORATION, TOKYO (JP)

(72) Inventors: TETSURO KUWAYAMA, TOKYO (JP); KAZUKI IKESHITA, TOKYO (JP); TAKANORI FUKAZAWA, TOKYO (JP)

(21) Appl. No.: 17/296,680

(22) PCT Filed: Nov. 5, 2019
(86) PCT No.: PCT/JP2019/043195

§ 371 Date: May 25, 2021

(51) Int. Cl. A61B 1/00; A61B 1/045; A61B 5/026; A61B 1/06

(52) U.S. Cl. CPC A61B 1/00009; A61B 1/0661; A61B 5/026; A61B 1/045

(57) ABSTRACT

A medical system (1) includes: an irradiation means (2) that irradiates a subject with coherent light; an imaging means (3) that captures an image of reflected light of the coherent light from the subject; an acquiring means (411) that acquires a speckle image from the imaging means (3); a calculating means (412) that performs, for each pixel of the speckle image, on the basis of luminance values of that pixel and surrounding pixels, statistical processing and calculation of a predetermined index value; a determining means (413) that determines, for the each pixel, whether or not a mean of the luminance values used in the calculation of the index value is in a predetermined range; a generating means (414) that generates a predetermined image on the basis of the index values; and a display control means (415) that identifiably displays, in displaying the predetermined image on a display means, a portion of pixels each having a mean of the luminance values, the mean being outside the predetermined range.

### FIELD

The present disclosure relates to medical systems, information processing devices, and information processing methods.

### BACKGROUND

Speckle imaging technology, which enables constant observation of bloodstream or lymph stream, has been developed in the medical field, for example. Speckling is a phenomenon where a spotty pattern is generated through reflection and interference of emitted coherent light due to microscopic roughness on a surface of a subject (a target), for example. On the basis of this speckling phenomenon, a bloodstream portion and a non-bloodstream portion in a living body that is a subject are able to be identified, for example.

Specifically, a bloodstream portion has a small speckle contrast value (hereinafter, also referred to as an “SC”) due to movement of red blood cells that reflect coherent light, for example, and a non-bloodstream portion has a large SC as the non-bloodstream portion is stationary overall. Therefore, bloodstream portions and non-bloodstream portions are able to be identified on the basis of a speckle contrast image generated using the SC of each pixel.

Index values calculated by statistical processing of speckles' luminance values may be, instead of SCs, for example: inverses of the SCs; squares of the inverses of the SCs; blur rates (BRs); square BRs (SBRs); or mean BRs (MBRs) (hereinafter, simply referred to as “index values”). Furthermore, values associated with cerebral blood flow (CBF) or cerebral blood volume (CBV) may be evaluated on the basis of these index values.

### CITATION LIST

### Patent Literature

Patent Literature 1: Japanese Unexamined Patent Application, Publication No. 2017-170064

### SUMMARY

### Technical Problem

By generating and displaying a given image (for example, an SC image) on the basis of index values, bloodstream is able to be evaluated (visually recognized) in, for example, bypass surgery for joining blood vessels together, clipping surgery on cerebral aneurysm, or brain tissue examination. When bloodstream in a blood vessel is being observed, for example, due to mirror reflection at a surface of the blood vessel, a part of the bloodstream may become higher in luminance and smaller in SC. When this happens, flow of the bloodstream in that part appears to be fast and this unevenness of the flow may give a false impression that the part has thrombus.

Furthermore, in bypass surgery, depending on how a subject is exposed to illumination, some blood vessels may become higher or lower in luminance. In that case, the blood flow may appear less or more than the actual blood flow in a displayed given image and this may lead to incorrect determination.

Similarly, in clipping surgery, aneurysm is oriented or shaped differently depending on the clip and may become higher or lower in luminance due to the change in the way illumination light is reflected, and thus a given image may be displayed with a blood flow different from the actual blood flow and this may also lead to incorrect determination.

Therefore, the present disclose proposes a medical system, an information processing device, and an information processing method enabling a portion to be identifiably displayed in a case where a predetermined image is generated by calculation of predetermined index values from a speckle image and the predetermined image is displayed, the portion having improper luminance used in the calculation of the index values.

### Solution to Problem

To solve the above-described problem, a medical system according to one aspect of the present disclosure comprises: an irradiation means that irradiates a subject with coherent light; an imaging means that captures an image of reflected light of the coherent light from the subject; an acquiring mean that acquires a speckle image from the imaging means; a calculating means that performs, for each pixel of the speckle image, on the basis of luminance values of that pixel and surrounding pixels, statistical processing and calculation of a predetermined index value; a determining means that determines, for the each pixel, whether or not a mean of the luminance values used in the calculation of the index value is in a predetermined range; a generating means that generates a predetermined image on the basis of the index values; and a display control means that identifiably displays, in displaying the predetermined image on a display means, a portion of pixels each having a mean of the luminance values, the mean being outside the predetermined range.

### BRIEF DESCRIPTION OF DRAWINGS

FIG. 1 is a diagram illustrating an example of a configuration of a medical system according to an embodiment of the present disclosure.

FIG. 2 is a diagram illustrating an example of a configuration of an information processing device according to the embodiment of the present disclosure.

FIG. 3 is a diagram illustrating an example of an SC image of a pseudo blood vessel.

FIG. 4 is a diagram illustrating relations between mean signal level and speckle contrast.

FIG. 5A is a schematic diagram illustrating a distribution of noise and a target signal having a proper mean luminance value.

FIG. 5B is a schematic diagram illustrating a distribution of noise and a target signal having a mean luminance value that is too small.

FIG. 5C is a schematic diagram illustrating a distribution of noise and a target signal having a mean luminance value that is too large.

FIG. 6 is a schematic diagram illustrating a proper range of mean luminance in the embodiment of the present disclosure.

FIG. 7 is a diagram illustrating a speckle image and an SC image in the embodiment of the present disclosure.

FIG. 8 is a flow chart illustrating processing by the information processing device according to the embodiment of the present disclosure.

FIG. 9 is a diagram illustrating an example of a schematic configuration of an endoscopic surgical system according to a first application example of the present disclosure.

FIG. 10 is a block diagram illustrating an example of a functional configuration of a camera head and a CCU illustrated in FIG. 9.

FIG. 11 is a diagram illustrating an example of a schematic configuration of a microscopic surgical system according to a second application example of the present disclosure.

FIG. 12 is a diagram illustrating how surgery is performed using the microscopic surgical system illustrated in FIG. 11.

### DESCRIPTION OF EMBODIMENTS

Embodiments of the present disclosure will be described in detail below on the basis of the drawings. Redundant explanation will be omitted as appropriate by assignment of the same reference sign to components that are the same in the following embodiments.

First of all, significance of the present invention will be described afresh. Evaluation of bloodstream is important in many cases in the medical field. For example, in a bypass operation in brain surgery, patency (bloodstream) is checked after blood vessels are joined together. Furthermore, in clipping surgery on aneurysm, flow of bloodstream into the artery is checked after clipping. For these purposes, bloodstream evaluation by angiography using an ultrasound Doppler blood flowmeter or an indocyanine green (ICG) agent has been performed, for example.

However, the ultrasound Doppler blood flowmeter measures bloodstream at a single point that the probe is brought into contact with and thus the overall bloodstream trend distribution in the surgical field cannot be known. Furthermore, there is a risk that evaluation needs to be performed by contact with the cerebrovascular vessel.

Furthermore, angiography using an ICG agent utilizes the ICG agent's characteristic of fluorescing due to near-infrared excitation light by combining with plasma protein in a living body, and is thus invasive observation involving administration of the agent. In addition, for bloodstream evaluation, the flow needs to be determined from a change happening immediately after the administration of the ICG agent, and thus the way of use is limited in terms of timing also.

Under such circumstances, speckle imaging technology is available as a bloodstream evaluation method for visualizing bloodstream without administration of a medical agent. For example, an optical device for perfusion evaluation in speckle imaging technology has been disclosed by Japanese Unexamined Patent Application, Publication No. 2017-170064. The principle of detecting movement (bloodstream) by utilization of speckles generated by laser is used therein. A case where speckle contrast (SC) is utilized as an index of movement detection will be described below, for example.

An SC is a value expressed by (standard deviation)/(mean value) of a light intensity distribution. In a portion having no movement, the light intensity is distributed from a locally bright portion to a locally dark portion of the speckle pattern, and thus the standard deviation of the intensity distribution is large and the SC (the degree of glare) is high. In contrast, in a portion having movement, the speckle pattern changes in association with the movement. If a speckle pattern is imaged in an observation system having a certain exposure time, the imaged speckle pattern is averaged and the SC (the degree of glare) becomes lower because the speckle pattern is changed over the exposure time. In particular, the larger the movement is, the more averaged the imaged speckle pattern is and thus the lower the SC becomes. Accordingly, the amount of movement is able to be known by evaluation of the SC.

This technique involves a method of performing statistical evaluation using luminance values of a pixel of interest and plural surrounding pixels (for example, 3×3 pixels or 5×5 pixels that are around the pixel of interest). Therefore, the mean of the luminance values (hereinafter, also referred to as the “mean luminance”) of the pixel of interest and plural surrounding pixels needs to be in a proper range (a predetermined range) for a proper index value to be calculated.

Therefore, a medical system, an information processing device, and an information processing method will be described below, the medical system, the information processing device, and the information processing method enabling a portion to be identifiably displayed in a case where a predetermined image is generated by calculation of predetermined index values from a speckle image and the predetermined image is displayed, the portion having improper luminance used in the calculation of the index values.

Configuration of Medical System According to Embodiment

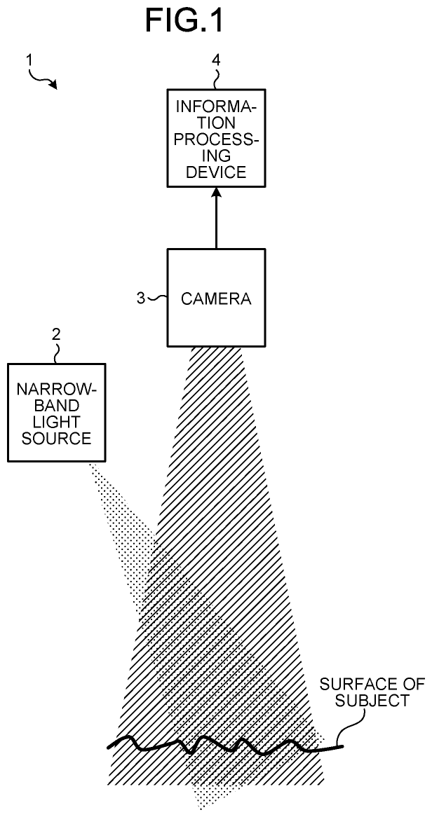

FIG. 1 is a diagram illustrating an example of a configuration of a medical system 1 according to an embodiment of the present disclosure. The medical system 1 according to the embodiment includes a narrow-band light source 2 (an irradiation means), a camera 3 (an imaging means), and an information processing device 4. Each of these components will be described in detail below.

(1) Light Source

The narrow-band light source 2 irradiates a subject with coherent light (for example, coherent near-infrared light, hereinafter, also simply referred to as “near-infrared light”). Coherent light refers to light having temporally unchanging and constant phase relation between light waves at any two points in a flux of the light and having complete coherence even if the flux of light is split by any method and the split parts are thereafter superimposed together again with a large optical path difference therebetween.

The coherent light output from the narrow-band light source 2 according to the present disclosure preferably has a wavelength of about 800 nm to 900 nm, for example. For example, if the wavelength is 830 nm, ICG observation and an optical system are able to be used in combination. That is, because near-infrared light having a wavelength of 830 nm is generally used in ICG observation, if near-infrared light having the same wavelength is also used in speckle observation, speckle observation is able to be performed without changing the optical system of the microscope enabling ICG observation.

The wavelength of the coherent light emitted by the narrow-band light source 2 is not limited to the above wavelength, and various other wavelengths may be used. For example, in a case where visible coherent light having a wavelength of 450 nm to 700 nm is used, laser used in projectors, for example, is able to be selected easily. Furthermore, in a case where an imager other than a Si-imager is adopted, coherent light having a wavelength of 900 nm or longer may be used. A case where near-infrared light having a wavelength of 830 nm is used as the coherent light will be described below as an example.

Furthermore, the type of the narrow-band light source 2 that emits the coherent light is not particularly limited so long as effects of the present techniques are not lost. Any one or combination selected from a group of an argon ion (Ar) laser, a helium-neon (He—Ne) laser, a dye laser, a krypton (Cr) laser, a semiconductor laser, and a solid-state laser that is a combination of a semiconductor laser and a wavelength conversion optical element, for example, may be used as the narrow-band light source 2 that emits laser light.

(2) Subject

There are various examples of subjects, but a subject including fluid, for example, is suitable. Speckles have a characteristic of being difficult to be generated from fluid. Therefore, if a subject including fluid is subjected to imaging using the medical system 1 according to the present disclosure, the boundary between a fluid portion and a non-fluid portion and the flow rate of the fluid portion are able to be found, for example.

More specifically, for example, a subject may be a living body including fluid that is blood. For example, surgery is able to be performed while checking the position of a blood vessel by using the medical system 1 according to the present disclosure in microscopic surgery or endoscopic surgery, for example. Therefore, safer and more precise surgery is able to be performed and this contributes to further advancement of the medical technology

(3) Imaging Device

The camera 3 captures an image of reflected light (scattered light) of near-infrared light from a subject. The camera 3 is, for example, an infrared (IR) imager for speckle observation. The camera 3 captures a speckle image acquired from the near-infrared light.

(4) Information Processing Device

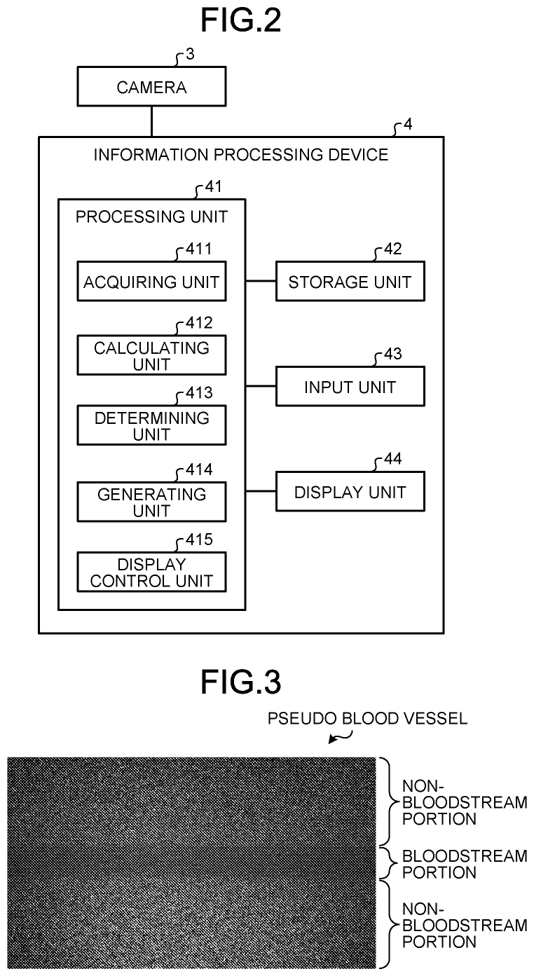

The information processing device 4 will be described next by reference to FIG. 2. FIG. 2 is a diagram illustrating an example of a configuration of the information processing device 4 according to the embodiment of the present disclosure. The information processing device 4 is an image processing device and includes, as its main components, a processing unit 41, a storage unit 42, an input unit 43, and a display unit 44 (a display means).

The processing unit 41 is implemented by, for example, a central processing unit (CPU) and includes, as its main components, an acquiring unit 411 (an acquiring means), a calculating unit 412 (a calculating means), a determining unit 413 (a determining means), a generating unit 414 (a generating means), and a display control unit 415 (a display control means).

The acquiring unit 411 acquires a speckle image from the camera 3. Furthermore, the calculating unit 412 calculates, for each pixel of the speckle image, a predetermined index value (for example, an SC) by performing statistical processing on the basis of luminance values of that pixel and its surrounding pixels.

A speckle contrast value of an i-th pixel (a pixel of interest) is able to be expressed by Equation (1) below.

Speckle contrast value of i-th pixel=(Standard deviation of intensities of i-th pixel and surrounding pixels)/(Mean of intensities of i-th pixel and surrounding pixels (1)

For each pixel, the determining unit 413 determines whether or not the mean of luminance values used in calculation of an index value is in a predetermined range. Furthermore, on the basis of the index values (for example, the SCs), the generating unit 414 generates a predetermined image (for example, an SC image).

The display control unit 415 displays the predetermined image on the display unit 44. Furthermore, in displaying the predetermined image on a display means, the display control unit 415 identifiably displays a portion with pixels having mean luminance values outside the predetermined range. Furthermore, in displaying the predetermined image on the display means, the display control unit 415 may display the portion with the pixels having the mean luminance values outside the predetermined range, such that whether their mean luminance values are smaller than a lower limit value of the predetermined range or larger than an upper limit value of the predetermined range is able to be identified. A portion having mean luminance values smaller than the lower limit value of the predetermined range may hereinafter be referred to as a “low luminance portion”, the mean luminance values being used in calculation of index values, and a portion having mean luminance values larger than the upper limit value of the predetermined range may hereinafter be referred to as a “high luminance portion”, the mean luminance values being used in calculation of index values.

Furthermore, in generating a predetermined image on the basis of index values, the generating unit 414 generates the predetermined image such that any of lightness, hue, and chroma of a predetermined color corresponds to the magnitude of the index value for each pixel. In this case, in displaying the predetermined image on a display means, the display control unit 415 identifiably displays a portion with pixels having mean luminance values outside a predetermined range by displaying the portion in a color other than that predetermined color.

The storage unit 42 stores various types of information, such as a speckle image acquired by the acquiring unit 411, a result of calculation performed by each unit of the processing unit 41, and various threshold values. A storage device external to the medical system 1 may be used, instead of this storage unit 42.

The input unit 43 is a means for a user to input information, and is, for example, a keyboard and a mouse.

Under control from the display control unit 415, the display unit 44 displays various types of information, such as a speckle image acquired by the acquiring unit 411, a result of calculation by each unit of the processing unit 41, and various threshold values. A display device external to the medical system 1 may be used, instead of this display unit 44.

An example of an SC image will now be described by reference to FIG. 3. FIG. 3 is a diagram illustrating an example of an SC image of a pseudo blood vessel. As illustrated by the example of the SC image in FIG. 3, many speckles are observed in a non-bloodstream portion and very few speckles are observed in a bloodstream portion.

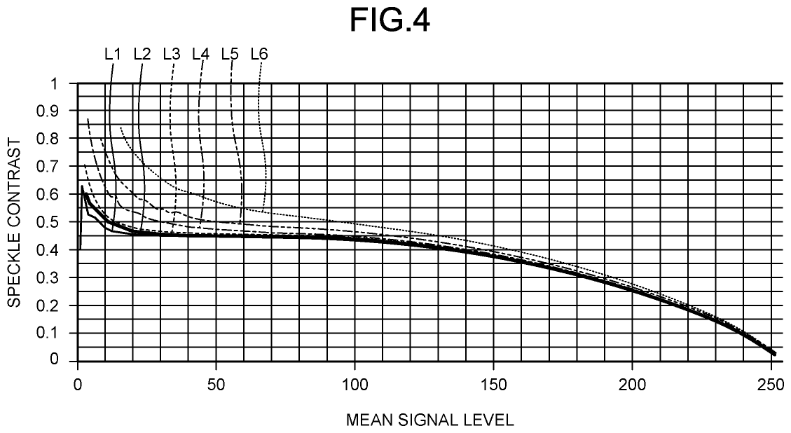

FIG. 4 is a diagram illustrating relations between mean signal level and speckle contrast. In FIG. 4, the horizontal axis represents the speckle contrast (SC) and the vertical axis represents the mean signal level (the mean luminance value). A stationary target was used herein as a subject, and SC for the same subject was analyzed at different quantities of illumination light.

A relational line L1 represents a relation between mean signal level and SC, for a predetermined gain (an amplification factor of the imaging element) in the camera 3. A relational line L2, a relational line L3, a relational line L4, a relational line L5, and a relational line L6 respectively represent relations between mean signal level and SC in cases where the gain is increased twofold each time from that for the relational line L1.

Basically, SC is desirably constant regardless of the quantity of illumination light. However, for all of the relational line L1 to relational line L6, when the mean signal level is low, the SC becomes larger than the actual value, and when the mean signal level is large, the SC becomes smaller than the actual value. This indicates that in displaying an SC image, identifiably displaying a portion having improper luminance used in the calculation of the SC is effective.

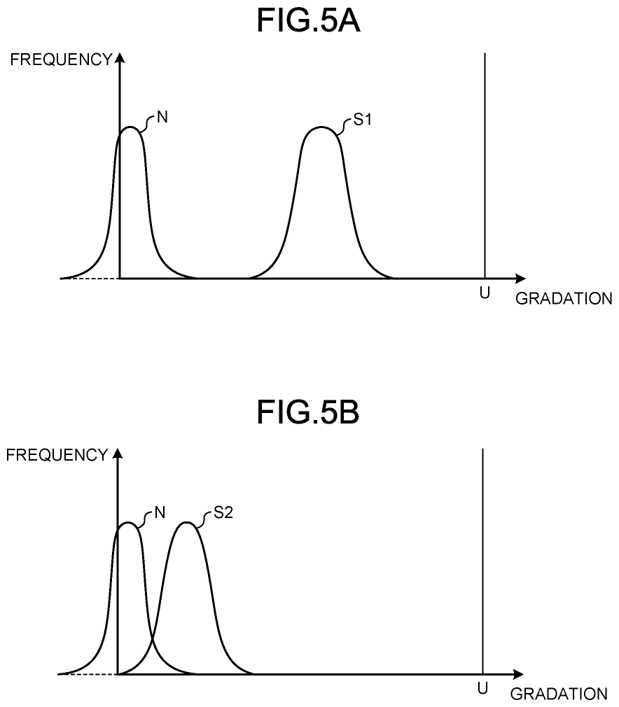

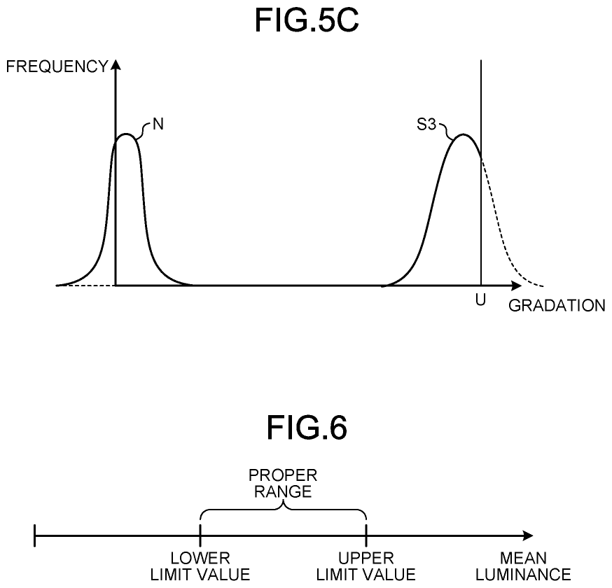

Relations between a target signal (a signal other than noise) and noise will be described next by reference to FIG. 5A to FIG. 5C. Firstly, FIG. 5A is a schematic diagram illustrating a distribution of noise and a target signal having a proper mean luminance value. In FIG. 5A to FIG. 5C, the horizontal axis represents gradation (luminance values: for example 0 to 255) and the vertical axis represents frequency.

In the state of FIG. 5A, a target signal S is significantly larger than noise N (that is, influence of the noise N is small), the target signal S has not reached the upper limit U (for example, 255) of the gradation, and thus the mean luminance value of the target signal can be said to be proper.

FIG. 5B is a schematic diagram illustrating a distribution of noise and a target signal having a mean luminance value that is too small. In the state of FIG. 5B, the target signal S is not significantly larger than the noise N, that is, influence of the noise N is large, and thus the mean luminance value of the target signal cannot be said to be proper. Specifically, the SC has a value larger than the actual value and indicating that the movement is smaller than the actual amount of movement.

FIG. 5C is a schematic diagram illustrating a distribution of noise and a target signal having a mean luminance value that is too large. In the state of FIG. 5B, the target signal S is significantly larger than the noise N (that is, influence of the noise N is small). However, the target signal S has reached the upper limit U of the gradation and the portion equal to or greater than the upper limit U is stuck to the upper limit U, and thus the mean luminance value and standard deviation value are different from the actual values and the mean luminance value of the target signal cannot be said to be proper. Specifically, the SC has a value smaller than the actual value and indicating that the movement is larger than the actual amount of movement.

Therefore, a proper SC is unable to be calculated if luminance is too high or too low. This is because luminance values are statistically processed in speckle imaging technology. The same applies to a case with other index values although SC has been described as an example.

A proper range of mean luminance of a pixel of interest and plural surrounding pixels for calculating an index value will be described next. FIG. 6 is a schematic diagram illustrating a proper range of mean luminance in the embodiment of the present disclosure. The proper range of mean luminance is from a predetermined lower limit value to a predetermined upper limit value.

The lower limit value is set on the basis of a standard deviation of noise in a speckle image, for example. The upper limit value is set on the basis of a gradation number (for example, 256) of luminance in the speckle image, for example.

More specifically, in a case where a value that is over a signal level considered to be proper by about ±5% is regarded as being in error (outside the proper range), the lower limit value and the upper limit value of the proper range may be set as follows, in consideration of relation between signal level and noise and relation to the number of operation bits.

Lower limit value: value of about 15 times the standard deviation of sensor noise

Upper limit value: value of about 40% of the gradation number

(For example, about 100 if the number of operation bits is 8 bits and the gradation number is 256 (=2 to the power of 8), and about 400 if the number of operation bits is 10 bits and the gradation number is 1024 (2 to the power of 10))

The above mentioned numerical values are just examples, and the embodiment is not limited to these examples. Noise is broadly divided into invariable noise and variable noise. Invariable noise is, for example, quantized noise, reading noise, or noise due to heat. Variable noise is, for example, shot noise. The lower limit value and upper limit value of the proper range may be modified as appropriate in consideration of these various types of noise and quantity of illumination light, for example.

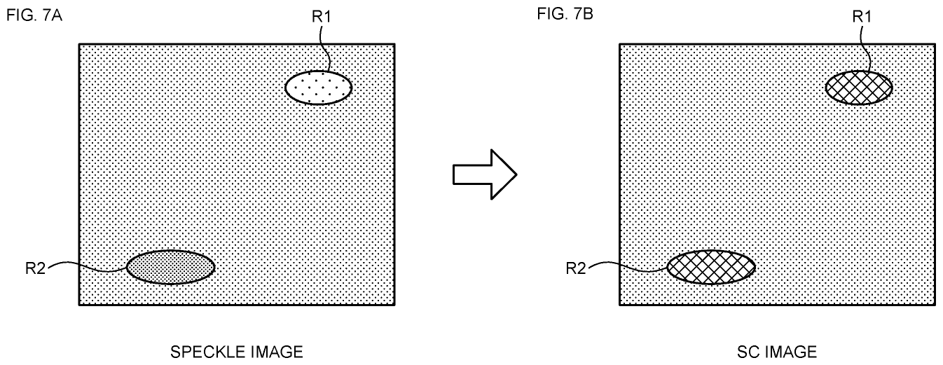

An example, in which a low luminance portion and a high luminance portion are identifiably displayed in an SC image that is an example of a predetermined image, will be described next by reference to FIG. 7. FIG. 7 is a diagram illustrating a speckle image (FIG. 7(a)) and an SC image (FIG. 7(b)) in the embodiment of the present disclosure.

For example, as illustrated in FIG. 7(a), an area R1 is a high luminance portion and an area R2 is a low luminance portion in the speckle image. In this case, as illustrated in FIG. 7(b), the areas R1 and R2 are identifiably displayed as being in error, in the SC image. Specifically, for example, if gradation display having white and black at ends is performed according to the magnitude of SC in the SC image, the areas R1 and R2 may be displayed in a color other than white and black (for example, red or blue).

Furthermore, for example, if gradation display having red and blue at ends is performed according to the magnitude of SC in the SC image, the areas R1 and R2 may be displayed in a color other than red and blue (for example, white or black). A user is thereby able to readily recognize the high luminance portion and low luminance portion by looking at such display.

Furthermore, the high luminance portion and the low luminance portion may be identifiably displayed in different colors. As a result, a user is able to readily distinguish between and deal with these portions.

The ways of using colors described above are just examples, the embodiment is not limited to these examples, and any way of using colors is possible as long as a portion having mean luminance values outside the proper range is able to be readily recognized.

Processing by Information Processing Device According to Embodiment

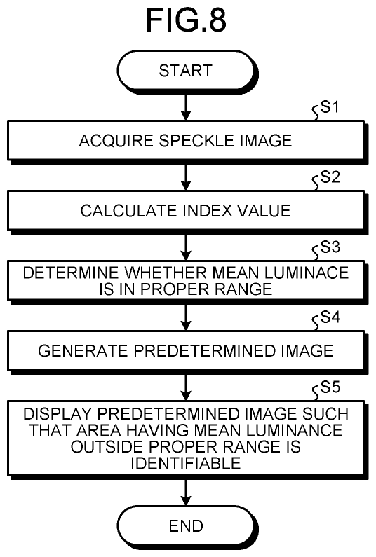

Processing by the information processing device 4 according to the embodiment of the present disclosure will be described next by reference to FIG. 8. FIG. 8 is a flow chart illustrating processing by the information processing device 4 according to the embodiment of the present disclosure.

Firstly, at Step S1, the acquiring unit 411 acquires a speckle image from the camera 3.

Next, at Step S2, the calculating unit 412 calculates, for each pixel of the speckle image, a predetermined index value (for example, an SC) by performing statistical processing on the basis of luminance values of that pixel and its surrounding pixels.

Subsequently, at Step S3, the determining unit 413 determines, for each pixel, whether or not a mean of the luminance values used in the calculation of the index value is in a predetermined range.

Next, at Step S4, the generating unit 414 generates a predetermined image (for example, an SC image) on the basis of the index values.

Subsequently, at Step S5, the display control unit 415 displays the predetermined image on the display unit 44, such that a portion (an area) of pixels having a mean luminance value that is outside the predetermined range is able to be identified.

As described above, in a case where a predetermined image is generated by calculation of predetermined index values from a speckle image and that predetermined image is displayed, the information processing device 4 of the embodiment enables a portion to be identifiably displayed, the portion having improper luminance used in the calculation of the index values.

Therefore, evaluation values that are more correct are able to be presented in speckle imaging technology and thus operators are able to be prevented from making determinations on the basis of incorrect information, for example.

Furthermore, when a high luminance portion and a low luminance portion are identifiably displayed, an operator is able to understand the situation more adequately.

### First Application Example

Techniques according to the present disclosure are applicable to various products. For example, techniques according to the present disclosure may be applied to endoscopic surgical systems.

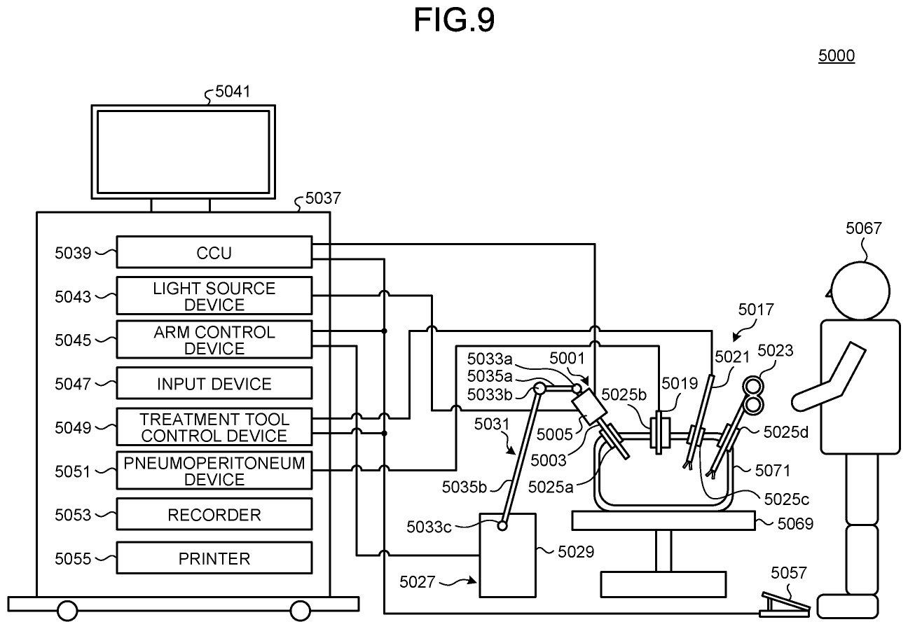

FIG. 9 is a diagram illustrating an example of a schematic configuration of an endoscopic surgical system 5000, to which techniques according to the present disclosure may be applied. FIG. 9 illustrates how an operator (a medical doctor) 5067 is performing surgery on a patient 5071 who is on a patient bed 5069, by using the endoscopic surgical system 5000. As illustrated, the endoscopic surgical system 5000 includes an endoscope 5001, other treatment tools 5017, a support arm device 5027 that supports the endoscope 5001, and a cart 5037 where various devices for endoscopic surgery are mounted.

In endoscopic surgery, plural tubular perforating devices called trocars 5025a to 5025d are tapped into an abdominal wall, instead of performing an abdominal section by cutting the abdominal wall. A lens barrel 5003 of the endoscope 5001 and the other treatment tools 5017 are inserted from the trocars 5025a to 5025d into a body cavity of the patient 5071. In the example illustrated, the other treatment tools 5017 that are a pneumoperitoneum tube 5019, an energy treatment tool 5021, and forceps 5023 are inserted into the body cavity of the patient 5071. Furthermore, the energy treatment tool 5021 is a treatment tool for performing incision and peeling of tissue or sealing of a blood vessel, for example, by using high-frequency electric current or ultrasound vibration. However, the treatment tools 5017 illustrated are just examples, and various treatment tools generally used in endoscopic surgery, such as, for example, tweezers and retractors, may be used as the treatment tools 5017.

An image of a surgical site in the body cavity of the patient 5071 captured by the endoscope 5001 is displayed on a display device 5041. The operator 5067 performs treatment, such as, for example, excision of an affected part, by using the energy treatment tool 5021 and forceps 5023, while looking, in real time, at the image of the surgical site displayed on the display device 5041. The pneumoperitoneum tube 5019, the energy treatment tool 5021, and the forceps 5023 are held up by the operator 5067 or an assistant, for example, during surgery, although illustration thereof in the drawings has been omitted.

Support Arm Device

The support arm device 5027 includes an arm unit 5031 that extends from a base unit 5029. In the illustrated example, the arm unit 5031 includes joints 5033a, 5033b, and 5033c and links 5035a and 5035b, and is driven by control from an arm control device 5045. The endoscope 5001 is supported by the arm unit 5031 and position and posture of the endoscope 5001 are controlled by the arm unit 5031. The position of the endoscope 5001 is thereby able to be stably locked.

Endoscope

The endoscope 5001 includes the lens barrel 5003 having a portion to be inserted into the body cavity of the patient 5071, the portion having a predetermined length from a distal end of the lens barrel 5003, and a camera head 5005 connected to a proximal end of the lens barrel 5003. In the example illustrated, the endoscope 5001 is configured as a so-called rigid endoscope having the lens barrel 5003 that is rigid, but the endoscope 5001 may be configured as a so-called flexible endoscope having the lens barrel 5003 that is flexible.

An opening with an objective lens fitted in the opening is provided at a distal end of the lens barrel 5003. A light source device 5043 is connected to the endoscope 5001, and light generated by the light source device 5043 is guided to the distal end of the lens barrel by a light guide provided to extend through the lens barrel 5003 and is emitted to an observation target (a subject) in the body cavity of the patient 5071 via the objective lens. The endoscope 5001 may be a direct viewing endoscope, an oblique viewing endoscope, or a side viewing endoscope.

An optical system and an imaging element are provided inside the camera head 5005, and reflected light (observation light) from the observation target is condensed by the optical system to the imaging element. The observation light is photoelectrically converted by the imaging element and an electric signal corresponding to the observation light, that is, an image signal corresponding to an observation image, is generated. The image signal is transmitted to a camera control unit (CCU) 5039 as RAW data. The camera head 5005 has a function of adjusting the magnification and focal length by driving its optical system as appropriate.

Plural imaging elements may be provided in the camera head 5005 to enable stereopsis (3D display), for example. In this case, plural relay optical systems are provided inside the lens barrel 5003 to guide observation light to each of the plural imaging elements.

Various Devices Mounted on Cart

The CCU 5039 includes a central processing unit (CPU) or a graphics processing unit (GPU), for example, and integrally controls operation of the endoscope 5001 and the display device 5041. Specifically, the CCU 5039 performs various types of image processing, such as, for example, development processing (demosaicing processing), for displaying an image based on an image signal received from the camera head 5005. The CCU 5039 provides the image signal that has been subjected to the image processing, to the display device 5041. Furthermore, the CCU 5039 transmits a control signal to the camera head 5005 to control driving of the camera head 5005. The control signal may include information related to imaging conditions, such as the magnification and focal length.

The display device 5041 displays, under control from the CCU 5039, an image based on the image signal that has been subjected to the image processing by the CCU 5039. If the endoscope 5001 is compatible with high resolution imaging, such as, for example, 4K (3840 horizontal pixels×2160 vertical pixels) or 8K (7680 horizontal pixels×4320 vertical pixels) imaging, and/or is compatible with 3D display, a display device that is capable of high resolution display and/or capable of 3D display is used as the display device 5041 correspondingly thereto. If the display device 5041 is compatible with high resolution imaging, such as 4K or 8K imaging, a greater sense of immersion is able to be obtained by use of a display device having a size of 55 inches or more as the display device 5041. Furthermore, plural display devices 5041 having different resolutions and sizes may be provided according to the intended use.

The light source device 5043 is formed of a light source, such as, for example, a light emitting diode (LED), and supplies irradiation light for imaging a surgical site, to the endoscope 5001.

The arm control device 5045 includes a processor, such as, for example, a CPU, and controls driving of the arm unit 5031 of the support arm device 5027 according to a predetermined control method by operating according to a predetermined program.

An input device 5047 is an input interface for the endoscopic surgical system 5000. A user is able to input various types of information and instructions to the endoscopic surgical system 5000 via the input device 5047. For example, the user inputs various types of information related to surgery, such as body information on a patient and a surgical method of the surgery, via the input device 5047. Furthermore, for example, the user inputs, via the input device 5047, an instruction to drive the arm unit 5031, an instruction to change imaging conditions for the endoscope 5001 (the type of irradiation light, magnification, and focal length, for example), and an instruction to drive the energy treatment tool 5021, for example.

The type of the input device 5047 is not limited, and the input device 5047 may be any of various known input devices. For example, a mouse, a keyboard, a touch panel, a switch, a foot switch 5057, and/or a lever may be used as the input device 5047. If a touch panel is used as the input device 5047, the touch panel may be provided on a display screen of the display device 5041.

Or, the input device 5047 is a device worn by a user, such as, for example, a spectacle-type wearable device or a head mounted display (HMD), and various types of input are performed according to the user's gestures or lines of sight detected by this device. Furthermore, the input device 5047 includes a camera that is capable of detecting movement of the user and various types of input are performed according to the user's gestures or lines of sight detected from a video captured by the camera. In addition, the input device 5047 includes a microphone capable of collecting voice of the user and various types of input are performed according to the voice via the microphone. As described above, by the input device 5047 being configured to be capable of inputting various types of information in a non-contact manner, in particular, a user in a clean area (for example, the operator 5067) is able to operate a device in a dirty area in a non-contact manner. Furthermore, because a user is able to operate a device without releasing the user's hand from a treatment tool being held by the user, convenience for the user is improved.

A treatment tool control device 5049 controls driving of the energy treatment tool 5021 for tissue cauterization or incision or sealing of a blood vessel, for example. A pneumoperitoneum device 5051 feeds gas into the body cavity of the patient 5071 via the pneumoperitoneum tube 5019 to inflate the body cavity for the purpose of obtaining a field of view for the endoscope 5001 and obtaining working space for the operator. A recorder 5053 is a device that is capable of recording various types of information related to surgery. A printer 5055 is a device that is capable of printing various types of information related to surgery, in various formats, such as text, images, or graphs.

Components that are particularly characteristic in the endoscopic surgical system 5000 will be described in more detail below.

Support Arm Device

The support arm device 5027 includes: the base unit 5029 that is a pedestal; and the arm unit 5031 that extends from the base unit 5029. In the illustrated example, the arm unit 5031 includes the plural joints 5033a, 5033b, and 5033c, and the plural links 5035a and 5035b connected to each other by the joint 5033b, but in FIG. 9, for simplification, a simplified configuration of the arm unit 5031 is illustrated. In fact, the shapes, numbers, and arrangements of the joints 5033a to 5033c and links 5035a and 5035b and the orientations of the rotational axes of the joints 5033a and 5033c, for example, are able to be set as appropriate, such that the arm unit 5031 has a desired number of degrees of freedom. For example, the arm unit 5031 may suitably be configured to have six degrees or more of freedom. The endoscope 5001 is thereby able to be moved freely in a movable range of the arm unit 5031 and the lens barrel 5003 of the endoscope 5001 is thus able to be inserted into the body cavity of the patient 5071 from a desired direction.

The joints 5033a and 5033c each have an actuator provided therefor, and are each configured to be rotatable about a predetermined rotational axis by being driven by the actuator. The driving of the actuators are controlled by the arm control device 5045, and the angles of rotation of the joints 5033a to 5033c are thereby controlled and driving of the arm unit 5031 is thereby controlled. As a result, position and posture of the endoscope 5001 are able to be controlled. In this control, the arm control device 5045 may control driving of the arm unit 5031 by any of various known control methods, such as force control or position control.

For example, by the operator 5067 inputting an operation as appropriate via the input device 5047 (including the foot switch 5057), the driving of the arm unit 5031 may be controlled appropriately by the arm control device 5045 according to the input of the operation and the position and posture of the endoscope 5001 may be controlled. Through this control, the endoscope 5001 at a distal end of the arm unit 5031 may be moved from any position to any other position and fixedly supported thereafter at that other position. The arm unit 5031 may be operated by a so-called master-slave method. In this case, the arm unit 5031 may be remotely operated by a user via the input device 5047 placed at a location away from the surgery room.

Furthermore, in a case where force control is applied, the arm control device 5045 may perform so-called power-assisted control in which the actuators of the joints 5033a to 5033c are driven such that the arm unit 5031 moves smoothly following external force received from a user. As a result, when moving the arm unit 5031 while directly touching the arm unit 5031, the user is able to move the arm unit 5031 with a comparatively light force. Therefore, the user is able to move the endoscope 5001 more intuitively by easier operation and convenience for the user is thus able to be improved.

In endoscopic surgery, the endoscope 5001 has generally been supported by a medical doctor, called a scopist. In contrast, by using the support arm device 5027, position of the endoscope 5001 is able to be locked more infallibly regardless of human intervention, and an image of a surgical site is thus able to be acquired stably and surgery is thus able to be performed smoothly.

The arm control device 5045 is not necessarily provided on the cart 5037. Furthermore, the arm control device 5045 is not necessarily a single device. For example, the arm control device 5045 may be provided in each of the joints 5033a to 5033c of the arm unit 5031 of the support arm device 5027, and driving of the arm unit 5031 may be controlled by mutual cooperation among the plural arm control devices 5045.

Light Source Device

The light source device 5043 supplies irradiation light for capturing an image of a surgical site, to the endoscope 5001. The light source device 5043 includes, for example, a white light source formed of, for example, an LED, a laser light source, or any combination of LEDs and laser light sources. In a case where the white light source is formed of a combination of RGB laser light sources, output intensity and output timing for each color (each wavelength) are able to be controlled highly accurately, and white balance of an image captured is thus able to be adjusted in the light source device 5043. Furthermore, in this case, an observation target is time-divisionally irradiated with laser light from each of the RGB laser light sources, driving of the imaging element in the camera head 5005 is controlled in synchronization with the irradiation timing, and images respectively corresponding to R, G, and B are thereby able to be captured time-divisionally. By this method, a color image is able to be acquired without provision of a color filter in the imaging element.

Furthermore, driving of the light source device 5043 may be controlled to change intensity of output light per predetermined time period. Images are time-divisionally acquired by controlling driving of the imaging element in the camera head 5005 in synchronization with the timing of that change in the intensity of light, these images are composited, and an image having a high dynamic range without so-called underexposure and overexposure is thereby able to be generated.

Furthermore, the light source device 5043 may be configured to be capable of supplying light of a predetermined wavelength band corresponding to special light observation. In special light observation, so-called narrow-band imaging is performed, the narrow-band imaging including imaging a predetermined tissue of a blood vessel in a surface layer of a mucous membrane, for example, at high contrast by: utilization of wavelength dependence of light absorption in body tissues, for example; and irradiation with light of a narrower band than that of irradiation light (that is, white light) for normal observation. Or, in special light observation, fluorescence observation, in which an image is acquired from fluorescence generated by irradiation with excitation light, may be performed. Fluorescence observation may involve, for example: observation of fluorescence from a body tissue irradiated with excitation light (autofluorescence observation); or acquisition of a fluorescent image by local injection of a reagent, such as indocyanine green (ICG), into a body tissue and irradiation of the body tissue with excitation light corresponding to a fluorescence wavelength of that reagent. The light source device 5043 may be configured to be capable of supplying narrow-band light and/or excitation light corresponding to such special light observation.

Camera Head and CCU

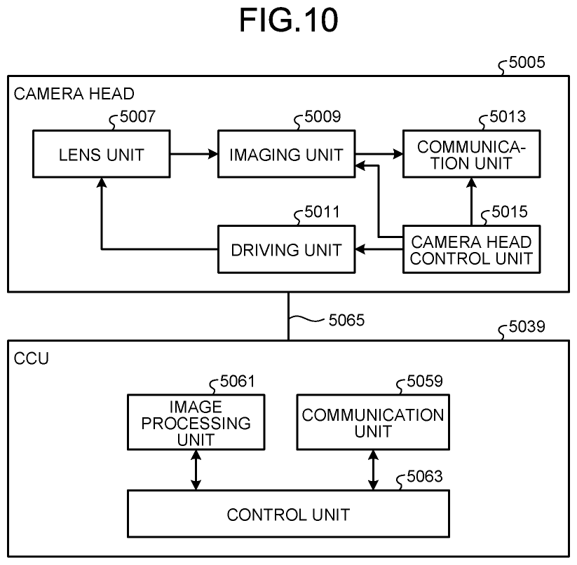

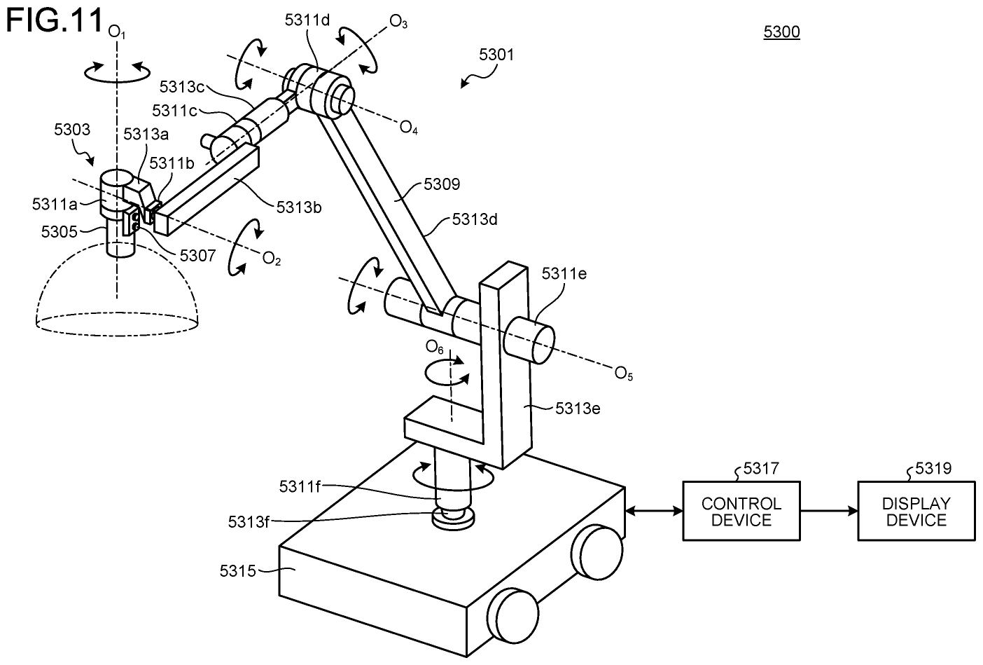

Functions of the camera head 5005 of the endoscope 5001 and the CCU 5039 will be described in more detail by reference to FIG. 10. FIG. 10 is a block diagram illustrating an example of a functional configuration of the camera head 5005 and the CCU 5039 illustrated in FIG. 9.

As illustrated in FIG. 10, the camera head 5005 has, as its functions, a lens unit 5007, an imaging unit 5009, a driving unit 5011, a communication unit 5013, and a camera head control unit 5015. Furthermore, the CCU 5039 has, as its functions, a communication unit 5059, an image processing unit 5061, and a control unit 5063. The camera head 5005 and the CCU 5039 are connected by a transmission cable 5065 to be communicable in both directions.

A functional configuration of the camera head 5005 will be described first. The lens unit 5007 is an optical system provided in a portion connected to the lens barrel 5003. Observation light taken in from the distal end of the lens barrel 5003 is guided to the camera head 5005 and enters the lens unit 5007. The lens unit 5007 is formed of a combination of plural lenses including a zoom lens and a focus lens. Optical properties of the lens unit 5007 are adjusted to condense observation light onto a light receiving surface of an imaging element in the imaging unit 5009. Furthermore, the zoom lens and focus lens are configured such that their position on the optical axis is movable for adjustment of the magnification and focus of an image captured.

The imaging unit 5009 includes the imaging element and is arranged downstream from the lens unit 5007. Observation light that has passed through the lens unit 5007 is condensed onto the light receiving surface of the imaging element, and an image signal corresponding to the observed image is generated by photoelectric conversion. The image signal generated by the imaging unit 5009 is provided to the communication unit 5013.

The imaging element used to form the imaging unit 5009 is, for example, a complementary metal oxide semiconductor (CMOS) image sensor having a Bayer array and capable of color imaging. The imaging element used may be capable of capturing images of high resolutions of 4K or more, for example. Acquisition of a high resolution image of a surgical site enables the operator 5067 to understand the state of the surgical site in more detail and to proceed with the surgery more smoothly.

Furthermore, the imaging element forming the imaging unit 5009 is configured to have a pair of imaging elements for respectively acquiring image signals for a right eye and a left eye corresponding to 3D display. The 3D display enables the operator 5067 to more accurately perceive the depth of a body tissue in a surgical site. In a case where the imaging unit 5009 is configured to be of the multi-element type, plural lens units 5007 are also provided correspondingly to the imaging elements.

Furthermore, the imaging unit 5009 is not necessarily provided in the camera head 5005. For example, the imaging unit 5009 may be provided inside the lens barrel 5003, immediately behind the objective lens.

The driving unit 5011 is formed of an actuator, and under control from the camera head control unit 5015, the driving unit 5011 moves the zoom lens and focus lens of the lens unit 5007 by a predetermined distance along the optical axis. The magnification and focus of an image captured by the imaging unit 5009 is thereby able to be adjusted as appropriate.

The communication unit 5013 is formed of a communication device for transmitting and receiving various types of information to and from the CCU 5039. The communication unit 5013 transmits, via the transmission cable 5065, an image signal acquired from the imaging unit 5009, the image signal being RAW data. In this transmission, for displaying a captured image of a surgical site at low latency, the image signal is preferably transmitted by optical communication. This is because surgery is performed while the operator 5067 is observing the state of an affected part from a captured image, and for safer and more infallible surgery, a moving image of a surgical site is desired to be displayed in real time whenever possible. In a case where optical communication is performed, a photoelectric conversion module for converting an electric signal into an optical signal is provided in the communication unit 5013. The image signal is converted into the optical signal by the photoelectric conversion module and the optical signal is thereafter transmitted to the CCU 5039 via the transmission cable 5065.

Furthermore, the communication unit 5013 receives, from the CCU 5039, a control signal for controlling driving of the camera head 5005. The control signal includes information related to imaging conditions, such as, for example, information specifying a frame rate of a captured image, information specifying an exposure value for imaging, and/or information specifying a magnification and a focus of the captured image. The communication unit 5013 provides the control signal received, to the camera head control unit 5015. The control signal from the CCU 5039 may be transmitted by optical communication also. In this case, a photoelectric conversion module for converting an optical signal to an electric signal is provided in the communication unit 5013, the control signal is converted into an electric signal by the photoelectric conversion module, and the electric signal is thereafter provided to the camera head control unit 5015.

Imaging conditions, such as the frame rate, exposure value, magnification, and focus described above are automatically set by the control unit 5063 of the CCU 5039 on the basis of the image signal acquired. That is, so called, an autoexposure (AE) function, an autofocus (AF) function, and an auto white balance (AWB) function are installed in the endoscope 5001.

The camera head control unit 5015 controls driving of the camera head 5005 on the basis of a control signal received via the communication unit 5013 from the CCU 5039. For example, on the basis of information specifying a frame rate of a captured image and/or information specifying exposure for imaging, the camera head control unit 5015 controls driving of the imaging element in the imaging unit 5009. Furthermore, on the basis of information specifying a magnification and a focus of the captured image, for example, the camera head control unit 5015 moves the zoom lens and the focus lens in the lens unit 5007 via the driving unit 5011 as appropriate. The camera head control unit 5015 may further include a function of storing information for identifying the lens barrel 5003 and the camera head 5005.

Arranging components, such as the lens unit 5007 and the imaging unit 5009, in a sealed structure that is highly airtight and waterproof enables the camera head 5005 to have resistance to autoclave sterilization.

A functional configuration of the CCU 5039 will be described next. The communication unit 5059 is formed of a communication device for transmitting and receiving various types of information to and from the camera head 5005. The communication unit 5059 receives an image signal transmitted via the transmission cable 5065, from the camera head 5005. As described above, the image signal may be suitably transmitted by optical communication. In this case, a photoelectric conversion module for converting an optical signal into an electric signal is provided in the communication unit 5059 to enable the optical communication. The communication unit 5059 provides the image signal converted into an electric signal, to the image processing unit 5061.

Furthermore, the communication unit 5059 transmits, to the camera head 5005, a control signal for controlling driving of the camera head 5005. The control signal may be transmitted by optical communication also.

The image processing unit 5061 performs various types of image processing on an image signal that is RAW data transmitted from the camera head 5005. The image processing includes various types of known signal processing, such as, for example, development processing, image quality enhancing processing (band enhancement processing, super-resolution processing, noise reduction (NR) processing, and/or hand-shake correction processing, for example) and/or enlargement processing (electronic zooming processing). Furthermore, the image processing unit 5061 performs detection processing on the image signal for performing AE, AF, and AWB.

The image processing unit 5061 includes a processor, such as a CPU or a GPU, and the above described image processing and detection processing are executed by the processor operating according to a predetermined program. In a case where the image processing unit 5061 is formed of plural GPUs, the image processing unit 5061 divides information related to an image signal as appropriate and these plural GPUs perform image processing in parallel.

The control unit 5063 performs various types of control related to capturing of an image of a surgical site by the endoscope 5001 and display of the image captured. For example, the control unit 5063 generates a control signal for controlling driving of the camera head 5005. The control unit 5063 generates the control signal on the basis of input by a user if the user has input any imaging condition. Or, if the AE function, AF function, and AWB function have been installed in the endoscope 5001, the control unit 5063 generates the control signal by calculating the optimum exposure value, focal length, and white balance as appropriate according to results of the detection processing by the image processing unit 5061.

Furthermore, on the basis of an image signal that has been subjected to image processing by the image processing unit 5061, the control unit 5063 causes the display device 5041 to display an image of a surgical site. The control unit 5063 recognizes various objects in the image of the surgical site by using various image recognition techniques. For example, the control unit 5063 may recognize a treatment tool, such as forceps, a specific site in a living body, bleeding, and mist during use of the energy treatment tool 5021, for example, by detecting shapes of edges and colors, for example, of objects included in the image of the surgical site. In causing the display device 5041 to display the image of the surgical site, the control unit 5063 causes various types of surgical support information to be displayed with the various types of surgical support information superimposed on the image of the surgical site, by using results of that recognition. By presentation of the surgical support information to the operator 5067 through display of the image with the surgical support information superimposed on the image, the operator 5067 is able to proceed with the surgery more safely and infallibly.

The transmission cable 5065 that connects the camera head 5005 and the CCU 5039 to each other is an electric signal cable compatible with communication of electric signals, an optical fiber compatible with optical communication, or a composite cable of the electric signal cable and the optical fiber.

In the illustrated example, communication is performed by wire using the transmission cable 5065, but communication between the camera head 5005 and the CCU 5039 may be performed wirelessly. If the communication between the camera head 5005 and the CCU 5039 is performed wirelessly, the transmission cable 5065 does not need to be laid in the surgery room and thus the medical staff are able to avoid being hindered, by the transmission cable 5065, from moving in the surgery room.

An example of the endoscopic surgical system 5000 to which techniques according to the present disclosure are applicable has been described above. The endoscopic surgical system 5000 has been described herein as an example, but a system to which techniques according to the present disclosure are applicable is not limited to this example. For example, techniques according to the present disclosure may be applied to a diagnostic flexible endoscopic surgical system, or to a microscopic surgical system that will be described below as a second application example.

Techniques according to the present disclosure are suitably applicable to the endoscope 5001 among the components described above. Specifically, techniques according to the present disclosure are applicable to a case where a bloodstream portion and a non-bloodstream portion in an image of a surgical site in a body cavity of the patient 5071 captured by the endoscope 5001 are displayed to be visually recognizable on the display device 5041 easily. By application of techniques according to the present disclosure to the endoscope 5001, in displaying a predetermined image (for example, an SC image) generated by calculation of predetermined index values (for example, SC) from a speckle image, a portion in which the magnitudes of luminance used in the calculation of the index values are not proper is able to be identifiably displayed. As a result, the operator 5067 is able to be prevented from making incorrect determinations by looking at display different from the actual blood flow, and is thus able to perform surgery more safely.

### Second Application Example

Techniques according to the present disclosure may be applied to a microscopic surgical system used in so-called microsurgery performed while a microscopic site in a patient is being subjected to enlarged observation.

FIG. 11 is a diagram illustrating an example of a schematic configuration of a microscopic surgical system 5300 to which techniques according to the present disclosure are applicable. As illustrated in FIG. 11, the microscopic surgical system 5300 includes a microscope device 5301, a control device 5317, and a display device 5319. In the description of the microscopic surgical system 5300 below, a “user” means any member of the medical staff who use the microscopic surgical system 5300, such as an operator or an assistant.

The microscope device 5301 has a microscope unit 5303 for enlarged observation of an observation target (a surgical site of a patient), an arm unit 5309 that supports the microscope unit 5303 at a distal end of the arm unit 5309, and a base unit 5315 that supports a proximal end of the arm unit 5309.

The microscope unit 5303 includes a cylindrical portion 5305 that is approximately cylindrical, an imaging unit (not illustrated in the drawings) provided inside the cylindrical portion 5305, and an operating unit 5307 provided in a part of an outer circumferential area of the cylindrical portion 5305. The microscope unit 5303 is an electronic imaging microscope unit (a so-called video microscope unit) that electronically acquires a captured image through an imaging unit.

A cover glass that protects the imaging unit inside the cylindrical portion 5305 is provided on a plane of an opening at a lower end of the cylindrical portion 5305. Light from an observation target (hereinafter, also referred to as observation light) passes through the cover glass to be incident on the imaging unit inside the cylindrical portion 5305. A light source formed of, for example, a light emitting diode (LED) may be provided inside the cylindrical portion 5305, and for imaging, the observation target may be irradiated with light from the light source, via the cover glass.

The imaging unit includes an optical system that condenses observation light, and an imaging element that receives the observation light condensed by the optical system. The optical system is formed of a combination of plural lenses including a zoom lens and a focus lens, and optical properties of the optical system are adjusted to form an image of the observation light on a light receiving surface of the imaging element. By receiving and photoelectrically converting the observation light, the imaging element generates a signal corresponding to the observation light, that is, an image signal corresponding to an observation image. The imaging element used may be, for example, an imaging element having a Bayer array and capable of color imaging. The imaging element may be any of various know imaging elements, such as a complementary metal oxide semiconductor (CMOS) image sensor or a charge coupled device (CCD) image sensor. The image signal generated by the imaging element is transmitted as RAW data, to the control device 5317. This transmission of the image signal may be performed suitably by optical communication. This is because at the scene of surgery, surgery is performed while an operator is observing the state of an affected part from a captured image, and for safer and more infallible surgery, a moving image of a surgical site is desired to be displayed in real time whenever possible. By the transmission of the image signal through optical communication, the captured image is able to be displayed at low latency.

The imaging unit may have a driving system that moves the zoom lens and focus lens of its optical system along their optical axis. Appropriate movement of the zoom lens and focus lens by the driving system enables adjustment of the enlargement magnification of a captured image and the focal length in the imaging. Furthermore, various functions, such as an autoexposure (AE) function and an autofocus (AF) function, that are generally able to be included in electronic imaging microscope units may be installed in the imaging unit.

Furthermore, the imaging unit may be configured as a so-called single-element type imaging unit having a single imaging element or a so-called multi-element type imaging unit having plural imaging elements. If the imaging unit is of the multi-element type, image signals respectively corresponding to R, G, and B may be generated by the imaging elements, and a color image may be acquired by these image signals being composited, for example. Or, the imaging unit may be configured to have a pair of imaging elements for respectively acquiring image signals for a right eye and a left eye compatible with stereopsis (3D display). The 3D display enables an operator to more accurately perceive the depth of a body tissue in a surgical site. If the imaging unit is of the multi-element type, plural optical systems may be provided correspondingly to the respective imaging elements.

The operating unit 5307 is an input means that includes, for example, a cross lever or switches, and receives input of a user's operations. For example, the user is able to input an instruction to change the enlargement magnification of an observation image and a focal length to the observation target, via the operating unit 5307. The driving system of the imaging unit moving the zoom lens and focus lens as appropriate according to the instruction enables adjustment of the enlargement magnification and focal length. Furthermore, for example, the user is able to input an instruction to switch the operation modes (an all-free mode and a locked mode described later) of the arm unit 5309, via the operating unit 5307. In moving the microscope unit 5303, the user may move the microscope unit 5303 while holding the cylindrical portion 5305 by grasping the cylindrical portion 5305. Therefore, the operating unit 5307 is preferably provided at a position that is able to be easily operated with the user's finger while the user is grasping the cylindrical portion 5305 such that the user is able to operate the operating unit 5307 even when the user is moving the cylindrical portion 5305.

The arm unit 5309 is formed by plural links (a first link 5313a to a sixth link 5313f) being pivotably connected to each other by plural joints (a first joint 5311a to a sixth joint 5311f).

The first joint 5311a is approximately cylindrical, and supports, at a distal end (a lower end) thereof, an upper end of the cylindrical portion 5305 of the microscope unit 5303 pivotably on a rotational axis (a first axis O1) parallel to a central axis of the cylindrical portion 5305. The first joint 5311a may be formed such that the first axis O1 coincides with the optical axis of the imaging unit in the microscope unit 5303. As a result, by causing the microscope unit 5303 to pivot on the first axis O1, the field of view is able to be changed such that a captured image is rotated.

The first link 5313a fixedly supports, at a distal end thereof, the first joint 5311a. Specifically, the first link 5313a is a rod-like member having an approximate L-shape, a side at a distal end thereof extends in a direction orthogonal to the first axis O1, and the first link 5313a is connected to the first joint 5311a such that an end portion of that side abuts on an outer circumferential upper end portion of the first joint 5311a. The second joint 5311b is connected to an end portion of the other side of the approximate L-shape of the first link 5313a, the other side being at a proximal end of the approximate L-shape.

The second joint 5311b is approximately cylindrical, and supports, at a distal end thereof, a proximal end of the first link 5313a pivotably on a rotational axis (a second axis O2) orthogonal to the first axis O1. A distal end of the second link 5313b is fixedly connected to a proximal end of the second joint 5311b.

The second link 5313b is a bar-shaped member having an approximate L-shape, a side at the distal end thereof extends in a direction orthogonal to the second axis O2, and an end portion of that side is fixedly connected to the proximal end of the second joint 5311b. The third joint 5311c is connected to the other side of the approximate L-shape of the second link 5313b, the other side being at a proximal end of the approximate L-shape.

The third joint 5311c has an approximate cylindrical shape, and supports, at a distal end thereof, a proximal end of the second link 5313b pivotably on a rotational axis (a third axis O3) mutually orthogonal to the first axis O1 and the second axis O2. A distal end of the third link 5313c is fixedly connected to a proximal end of the third joint 5311c. By causing a distal end configuration including the microscope unit 5303 to pivot on the second axis O2 and third axis O3, the microscope unit 5303 is able to be moved such that position of the microscope unit 5303 in a horizontal plane is changed. That is, by controlling the rotation about the second axis O2 and third axis O3, the field of view of a capture image is able to be moved in a plane.

The third link 5313c is configured to be approximately cylindrical at a distal end of the third link 5313c, and the proximal end of the third joint 5311c is fixedly connected to the cylindrical distal end such that they both have approximately the same central axes. The third link 5313c has a prism shape at a proximal end thereof and the fourth joint 5311d is connected to an end portion of the third link 5313c.

The fourth joint 5311d has an approximate cylindrical shape, and supports, at a distal end thereof, the proximal end of the third link 5313c pivotably on a rotational axis (a fourth axis O4) orthogonal to the third axis O3. A distal end of the fourth link 5313d is fixedly connected to a proximal end of the fourth joint 5311d.

The fourth link 5313d is a bar-shaped member extending approximately linearly, extends orthogonally to the fourth axis O4, and is fixedly connected to the fourth joint 5311d such that an end portion of the fourth link 5313d at the distal end of the fourth link 5313d abuts on a side surface of the approximate cylindrical shape of the fourth joint 5311d. The fifth joint 5311e is connected to a proximal end of the fourth link 5313d.

The fifth joint 5311e has an approximate cylindrical shape, and supports, at a distal end thereof, the proximal end of the fourth link 5313d pivotably on a rotational axis (a fifth axis O5) parallel to the fourth axis O4. A distal end of the fifth link 5313e is fixedly connected to a proximal end of the fifth joint 5311e. The fourth axis O4 and fifth axis O5 are rotational axes enabling the microscope unit 5303 to move upward and downward. By causing the distal end configuration including the microscope unit 5303 to pivot on the fourth axis O4 and fifth axis O5, height of the microscope unit 5303, that is, distance between the microscope unit 5303 and an observation target, is able to be adjusted.

The fifth link 5313e is formed of a combination of: a first member having an approximate L-shape with a side extending in a vertical direction and the other side extending in a horizontal direction; and a second member that is rod-shaped and extends vertically downward from a portion of the first member, the portion extending in the horizontal direction. The proximal end of the fifth joint 5311e is fixedly connected to a part of a portion of the first member of the fifth link 5313e, the portion extending in the vertical direction, the part being in the vicinity of an upper end of that portion. The sixth joint 5311f is connected to a proximal end (a lower end) of the second member of the fifth link 5313e.

The sixth joint 5311f has an approximate cylindrical shape, and supports, at a distal end thereof, a proximal end of the fifth link 5313e on a rotational axis (sixth axis O6) parallel to the vertical direction. A distal end of the sixth link 5313f is fixedly connected to a proximal end of the sixth joint 5311f.

The sixth link 5313f is a rod-like member extending in the vertical direction and has a proximal end fixedly connected to an upper surface of the base unit 5315.

Rotational ranges of the first joint 5311a to the sixth joint 5311f are set as appropriate to enable desired movement of the microscope unit 5303. As a result, in the arm unit 5309 having the above described configuration, movement of three translational degrees of freedom and three rotational degrees of freedom, a total of six degrees of freedom, is able to be achieved for movement of the microscope unit 5303. As described above, by forming the arm unit 5309 to achieve six degrees of freedom for movement of the microscope unit 5303, position and posture of the microscope unit 5303 are able to be freely controlled in a movable range of the arm unit 5309. Therefore, a surgical site is able to be observed from any angle and surgery is able to be carried out more smoothly.

The illustrated configuration of the arm unit 5309 is just an example, and the number and forms (lengths) of the links forming the arm unit 5309, and the number, arrangement positions, and rotational axes of the joints, for example, may be designed as appropriate to enable desired freedom. For example, as described above, to freely move the microscope unit 5303, the arm unit 5309 is preferably configured to have six degrees of freedom, but the arm unit 5309 may be configured to have more degrees of freedom (that is, redundant degrees of freedom). If there are redundant degrees of freedom, in the arm unit 5309, posture of the arm unit 5309 is able to be changed in a state where position and posture of the microscope unit 5303 have been locked. Therefore, control that is more convenient for an operator is able to be achieved, the control including controlling posture of the arm unit 5309 such that the arm unit 5309 does not come in the view of an operator looking at the display device 5319, for example.

The first joint 5311a to sixth joint 5311f may each have, provided therein, a driving system, such as a motor, and an actuator having an encoder that detects the angle of rotation at the joint, for example. The control device 5317 controlling driving of the actuator provided in each of the first joint 5311a to sixth joint 5311f as appropriate enables control of posture of the arm unit 5309, that is, position and posture of the microscope unit 5303. Specifically, on the basis of information on the angle of rotation of each joint detected by the encoder, the control device 5317 is able to know the current posture of the arm unit 5309 and the current position and posture of the microscope unit 5303. By using these pieces of information known, the control device 5317 calculates a control value (for example, an angle of rotation or torque generated) for each joint to enable movement of the microscope unit 5303 according to input of an operation by a user, and drives the driving system of each joint according to the control value. The method of control of the arm unit 5309 by the control device 5317 is not limited, and any of various known control methods, such as force control or position control, may be used.

For example, an operator may input an operation as appropriate via an input device not illustrated in the drawings, driving of the arm unit 5309 may thereby be controlled as appropriate by the control device 5317 according to the input of the operation, and position and posture of the microscope unit 5303 may thereby be controlled. This control enables the microscope unit 5303 to be moved from any position to another position and to be thereafter fixedly supported at that position after the movement. An input device that is able to be operated even when an operator has a treatment tool in the operator's hand, such as, for example, a foot switch, is preferably used as the input device, in consideration of convenience for the operator. Furthermore, input of an operation may be performed in a non-contact manner, on the basis of gesture detection or line-of-sight detection using a wearable device or a camera provided in the surgery room. As a result, even a user belonging to a clean area is able to operate, with more degrees of freedom, a device belonging to a dirty area. Or, the arm unit 5309 may be operated by a so-called master-slave method. In this case, the arm unit 5309 may be remotely operated by a user via an input device placed at a location away from the surgery room.

Furthermore, in a case where force control is used, so-called power assist control may be used, the power assist control involving reception of external force from a user and driving of the actuators of the first joint 5311a to the sixth joint 5311f such that the arm unit 5309 is moved smoothly according to the external force. The user is thereby able to move the microscope unit 5303 with a comparative light force when grasping the microscope unit 5303 to directly move position of the microscope unit 5303. Therefore, the user is able to move the microscope unit 5303 more intuitively by easier operation, and convenience for the user is thus able to be improved.

Furthermore, driving of the arm unit 5309 may be controlled such that the arm unit 5309 performs pivot operation. Pivot operation is operation for moving the microscope unit 5303 such that the optical axis of the microscope unit 5303 constantly heads to a predetermined point (hereinafter, referred to as a pivot point) in a space. This pivot operation enables observation of the same observation position from various directions and thus enables more detailed observation of an affected part. If the microscope unit 5303 is configured to be unable to adjust its focal length, pivot operation is preferably performed in a state where the distance between the microscope unit 5303 and the pivot point has been fixed. In this case, the distance between the microscope unit 5303 and the pivot point may be adjusted to a fixed focal length of the microscope unit 5303. The microscope unit 5303 thereby moves on a hemispherical surface (schematically illustrated in FIG. 11) having a radius around the pivot point, the radius corresponding to the focal length, and a sharp captured image is thus able to be acquired even if the observation direction is changed. In contrast, if the microscope unit 5303 is configured to be able to adjust its focal length, pivot operation may be performed in a state where the distance between the microscope unit 5303 and the pivot point is variable. In this case, for example, on the basis of information on the angle of rotation of each joint detected by the encoder, the control device 5317 may calculate a distance between the microscope unit 5303 and the pivot point and automatically adjust the focal length of the microscope unit 5303 on the basis of a result of that calculation. Or, if an AF function is provided in the microscope unit 5303, every time the distance between the microscope unit 5303 and the pivot point is changed by pivot operation, the focal length may be automatically adjusted by that AF function.

Furthermore, brakes for restraining rotation of the first joint 5311a to the sixth joint 5311f may be provided in the first joint 5311a to the sixth joint 5311f. Operation of the brakes may be controlled by the control device 5317. For example, if position and posture of the microscope unit 5303 is desired to be fixed, the control device 5317 actuates the brakes of the joints. The posture of the arm unit 5309, that is, the position and posture of the microscope unit 5303 are thereby able to be fixed without the actuators being driven, and electric power consumption is thus able to be reduced. If position and posture of the microscope unit 5303 are desired to be moved, the control device 5317 may release the brakes of the joints and drive the actuators according to a predetermined control method.

Such operation of the brakes may be performed according to an operation input by a user via the operating unit 5307 described above. If the user wants to move the position and posture of the microscope unit 5303, the user operates the operating unit 5307 to release the brakes of the joints. The operation mode of the arm unit 5309 is thereby changed to a mode where each joint is able to be rotated freely (the all-free mode). Furthermore, if the user wants to fix the position and posture of the microscope unit 5303, the user operates the operating unit 5307 to actuate the brakes of the joints. The operation mode of the arm unit 5309 is thereby changed to a mode where rotation at each joint is restrained (the locked mode).

By controlling operation of the microscope device 5301 and the display device 5319, the control device 5317 integrally controls operation of the microscopic surgical system 5300. For example, by operating the actuators of the first joint 5311a to the sixth joint 5311f according to a predetermined control method, the control device 5317 controls driving of the arm unit 5309. Furthermore, for example, by controlling operation of the brakes of the first joint 5311a to the sixth joint 5311f, the control device 5317 changes the operation mode of the arm unit 5309. In addition, for example, by performing various types of signal processing on an image signal acquired by the imaging unit in the microscope unit 5303 of the microscope device 5301, the control device 5317 generates image data for display and causes the display device 5319 to display the image data. The signal processing may involve any of various known signal processing, such as, for example, development processing (demosaicing processing), image quality enhancing processing (band enhancement processing, super-resolution processing, noise reduction (NR) processing, and/or hand-shake correction processing, for example) and/or enlargement processing (that is, electronic zooming processing).

Communication between the control device 5317 and the microscope unit 5303, and communication between the control device 5317 and the first joint 5311a to the sixth joint 5311f may be wired communication or wireless communication. For wired communication, communication through electric signals may be performed, or optical communication may be performed. A transmission cable used in the wired communication may be an electric signal cable, an optical fiber, or a composite cable of the electric signal cable and optical fiber, correspondingly to a communication system for the wired communication. In contrast, for wireless communication, there is no need to lay a transmission cable in a surgery room, and thus the medical staff are able to avoid being hindered, by the transmission cable, from moving in the surgery room.

The control device 5317 may be a processor, such as a central processing unit (CPU) or a graphics processing unit (GPU); or a microcomputer or control board having both a processor and a storage element, such as a memory, for example. By the processor of the control device 5317 operating according to a predetermined program, the various functions described above are able to be implemented. In the illustrated example, the control device 5317 is provided as a device separate from the microscope device 5301, but the control device 5317 may be installed inside the base unit 5315 of the microscope device 5301 to be integrally formed with the microscope device 5301. Or, the control device 5317 may be formed of plural devices. For example, a microcomputer or a control board may be provided for each of the first joint 5311a to the sixth joint 5311f of the arm unit 5309, these microcomputers or control boards may be connected communicably to one another, and functions similar to those of the control device 5317 may thereby be implemented.

The display device 5319 is provided in a surgery room, and under control of the control device 5317, displays an image corresponding to image data generated by the control device 5317. That is, an image of a surgical site captured by the microscope unit 5303 is displayed on the display device 5319. The display device 5319 may display, instead of the image of the surgical site, or together with the image of the surgical site; various types of information related to the surgery, such as, for example, body information on the patient and information on the surgical method of the surgery. In that case, the display on the display device 5319 may be changed as appropriate through an operation by a user. Or, more than one display device 5319 may be provided, and an image of the surgical site and various types of information related to the surgery may be displayed on each of the plural display devices 5319. The display device 5319 used may be any of various known display devices, such as a liquid crystal display device, or an electroluminescence (EL) display device.

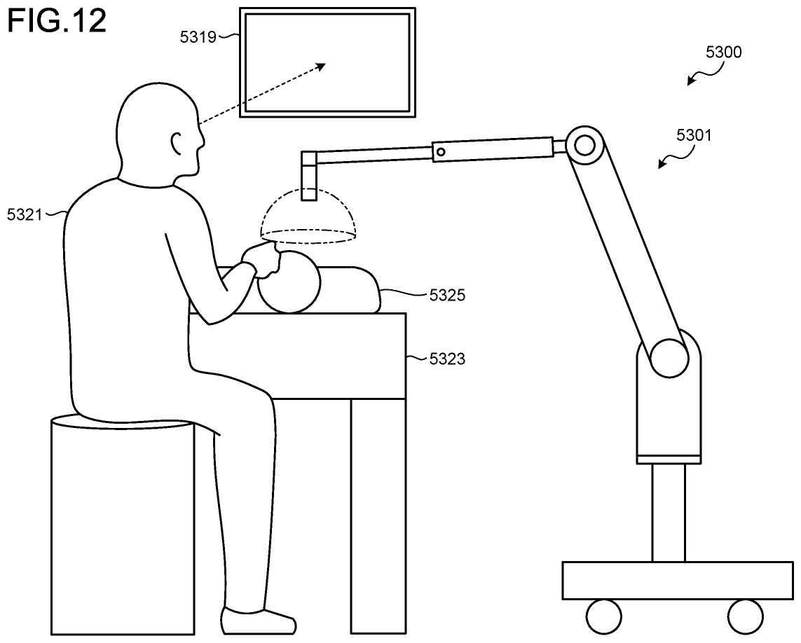

FIG. 12 is a diagram illustrating how surgery is performed using the microscopic surgical system 5300 illustrated in FIG. 11. FIG. 12 schematically illustrates how an operator 5321 is performing surgery on a patient 5325 who is on a patient bed 5323, by using the microscopic surgical system 5300. In FIG. 12, for simplification, illustration of the control device 5317 in the configuration of the microscopic surgical system 5300 has been omitted, and illustration of the microscope device 5301 has been simplified.

As illustrated in FIG. 2C, at the time of surgery, an enlarged image of a surgical site captured by the microscope device 5301 is displayed on the display device 5319 installed on a wall surface of a surgery room, by use of the microscopic surgical system 5300. The display device 5319 is installed at a position opposed to the operator 5321 and the operator 5321 performs various types of treatment, such as incision of an affected part, for example, on the surgical site, while observing the look of the surgical site from a video displayed on the display device 5319.

An example of the microscopic surgical system 5300 to which techniques according to the present disclosure may be applied has been described above. The microscopic surgical system 5300 has been described herein as an example, but a system to which techniques according to the present disclosure may be applied is not limited to this example. For example, the microscope device 5301 may also function as a support arm device that supports, at a distal end thereof, another observation device or another treatment tool, instead of the microscope unit 5303. Another observation device applicable, may be, for example, an endoscope. Furthermore, another treatment tool applicable may be, for example, forceps, tweezers, a pneumoperitoneum tube for pneumoperitoneum, or an energy treatment tool for tissue incision or sealing of a blood vessel by cauterization. By the support arm device supporting such an observation device or treatment tool, its position is able to be fixed more stably and the burden on the medical staff is able to be reduced, as compared to a case where a medical staff member supports the observation device or treatment tool by hand. Techniques according to the present disclosure may be applied to a support arm device that supports such a component other than a microscope unit.

Techniques according to the present disclosure may be suitably applied to the control device 5317 among the components described above. Specifically, techniques according to the present disclosure may be applied to a case where a bloodstream portion and a non-bloodstream portion in an image of a surgical site of the patient 5325 captured by the imaging unit in the microscope unit 5303 are displayed to be visually recognizable on the display device 5319 easily. By application of techniques according to the present disclosure to the control device 5317, in generating a predetermined image (for example, an SC image) by calculation of predetermined index values (for example, SC) from a speckle image and displaying the predetermined image, a portion in which the magnitudes of luminance used in the calculation of the index values are not proper is able to be displayed identifiably. As a result, the operator 5321 is able to be prevented from making incorrect determinations by looking at display different from the actual blood flow, and is thus able to perform surgery more safely.

### Third Application Example

In the above described embodiment and first and second application examples, in displaying a predetermined image (for example, an SC image), a portion in which magnitudes of luminance used in calculation of index values (for example, SC) are not proper is displayed identifiably. Putting this into use, such improper portions may be reduced.

For example, there is a method of correcting SC by measurement of a relation between luminance level and SC beforehand. Furthermore, there is also a method of calculating SC in consideration of noise of a camera. In addition, there is also a method of calculating SC in consideration of the influence of a state where, as illustrated in FIG. 5C, the mean luminance of a target signal S is too large, the target signal S reaches the upper limit U of gradation, and the portion equal to or larger than the upper limit U is stuck to the upper limit U. It is even more effective if the above described error display processing is performed after calculation or correction of SC by any of these methods.

### Fourth Application Example

Furthermore, there is also a method of providing feedback so as to reduce the area of error display. For example, if there is a low luminance portion, quantity of illumination light, exposure time, and gain may be increased such that the luminance is changed to be in a proper measurement range.

Furthermore, if there is a high luminance portion, the quantity of illumination light, exposure time, and gain may be reduced such that the luminance is changed to be in the proper measurement range.

Furthermore, if there are both a high luminance portion and a low luminance portion, the quantity of illumination light, exposure time, and gain, for example, may be adjusted such that their areas (the areas R1 and R2 in FIG. 7(a)) become closer to equal areas (or a predetermined ratio).

Furthermore, if there is error display in the center of a screen or in an area specified by a user, the quantity of illumination light, exposure time, and gain, for example, may be adjusted to eliminate the error display.

Using such feedback additionally is more effective as the area of error display is able to be reduced or error display in the area of interest (the center of the screen or an area specified by a user) is able to be eliminated, and the operator, for example, is thus able to acquire more information from a predetermined image (for example, an SC image).

The present techniques may also be provided in the following forms.

Some of embodiments and modified examples of the present disclosure have been described above, but the technical scope of the present disclosure is not limited to the above described embodiments and modified examples as is, and various modifications are possible without departing from the gist of the present disclosure. Furthermore, components of different ones of the embodiments and modified examples may be combined as appropriate.

Effects of the embodiments and application examples described in this specification are just examples, and they may have other effects without being limited to these examples.

Furthermore, for each of the above described embodiments, speckle contrast values have been described as an example of index values calculated by statistical processing of luminance values of speckles. However, without being limited to this example, the inverses of SCs, the squares of the inverses of the SCs, blur rates (BRs), square BRs (SBRs), or mean BRs (MBRs) may be used, for example. Furthermore, values associated with cerebral blood flow (CBF) or cerebral blood volume (CBV) may be evaluated on the basis of these index values.

Furthermore, the method of performing error display is not limited to the display using colors, and may be replaced by or combined with another method, such as display using text.

(1)

A medical system, comprising:

an irradiation means that irradiates a subject with coherent light;

an imaging means that captures an image of reflected light of the coherent light from the subject;

an acquiring mean that acquires a speckle image from the imaging means;

a calculating means that performs, for each pixel of the speckle image, on the basis of luminance values of that pixel and surrounding pixels, statistical processing and calculation of a predetermined index value;

a determining means that determines, for the each pixel, whether or not a mean of the luminance values used in the calculation of the index value is in a predetermined range;

a generating means that generates a predetermined image on the basis of the index values; and

a display control means that identifiably displays, in displaying the predetermined image on a display means, a portion of pixels each having a mean of the luminance values, the mean being outside the predetermined range.

(2)

The medical system according to (1), wherein the medical system is a microscopic surgical system or an endoscopic surgical system.

(3)

An information processing device, comprising:

an acquiring means that acquires a speckle image from an imaging means that captures an image of reflected light of coherent light with which a subject is irradiated;

a calculating means that performs, for each pixel of the speckle image, on the basis of luminance values of that pixel and surrounding pixels, statistical processing and calculation of a predetermined index value;

a determining means that determines, for the each pixel, whether or not a mean of the luminance values used in the calculation of the index value is in a predetermined range;

a generating means that generates a predetermined image on the basis of the index values; and

a display control means that identifiably displays, in displaying the predetermined image on a display means, a portion of pixels each having a mean of the luminance values, the mean being outside the predetermined range.

(4)

The information processing device according to (3), wherein the display control means displays, in displaying the predetermined image on the display means, the portion of the pixels each having the mean of the luminance values, the mean being outside the predetermined range, such that whether the mean is less than a lower limit value of the predetermined range or whether the mean is larger than an upper limit value of the predetermined range is able to be identified.

(5)

The information processing device according to (3) or (4), wherein

in generating the predetermined image on the basis of the index values, the generating means generates the predetermined image such that a predetermined color of the each pixel has lightness, hue, or chroma corresponding to a magnitude of the index value, and

in displaying the predetermined image on the display means, the display control means identifiably displays the portion of the pixels each having the mean of the luminance values, the mean being outside the predetermined range, by displaying the portion in a color other than the predetermined color.

(6)

The information processing device according to any one of (3) to (5), wherein an upper limit value of the predetermined range is set on the basis of a gradation number of luminance in the speckle image.

(7)

The information processing device according to any one of (3) to (6), wherein a lower limit value of the predetermined range is set on the basis of a standard deviation of noise in the speckle image.

(8)

An information processing method, including

an acquiring process of acquiring a speckle image from an imaging means that captures an image of reflected light of coherent light with which a subject is irradiated;

a calculating process of performing, for each pixel of the speckle image, on the basis of luminance values of that pixel and surrounding pixels, statistical processing and calculation of a predetermined index value;

a determining process of determining, for the each pixel, whether or not a mean of the luminance values used in the calculation of the index value is in a predetermined range;

a generating process of generating a predetermined image on the basis of the index values; and

a display control process of identifiably displaying, in displaying the predetermined image on a display means, a portion of pixels each having a mean of the luminance values, the mean being outside the predetermined range.

### REFERENCE SIGNS LIST

1 MEDICAL SYSTEM
2 NARROW-BAND LIGHT SOURCE
3 CAMERA
4 INFORMATION PROCESSING DEVICE
41 PROCESSING UNIT
42 STORAGE UNIT
43 INPUT UNIT
44 DISPLAY UNIT
411 ACQUIRING UNIT
412 CALCULATING UNIT
413 DETERMINING UNIT
414 GENERATING UNIT
413 DISPLAY CONTROL UNIT

1. A medical system, comprising:
an irradiation means that irradiates a subject with coherent light;
an imaging means that captures an image of reflected light of the coherent light from the subject;
an acquiring mean that acquires a speckle image from the imaging means;
a calculating means that performs, for each pixel of the speckle image, on the basis of luminance values of that pixel and surrounding pixels, statistical processing and calculation of a predetermined index value;
a determining means that determines, for the each pixel, whether or not a mean of the luminance values used in the calculation of the index value is in a predetermined range;
a generating means that generates a predetermined image on the basis of the index values; and
a display control means that identifiably displays, in displaying the predetermined image on a display means, a portion of pixels each having a mean of the luminance values, the mean being outside the predetermined range.

2. The medical system according to claim 1, wherein the medical system is a microscopic surgical system or an endoscopic surgical system.

3. An information processing device, comprising:
an acquiring means that acquires a speckle image from an imaging means that captures an image of reflected light of coherent light with which a subject is irradiated;
a calculating means that performs, for each pixel of the speckle image, on the basis of luminance values of that pixel and surrounding pixels, statistical processing and calculation of a predetermined index value;
a determining means that determines, for the each pixel, whether or not a mean of the luminance values used in the calculation of the index value is in a predetermined range;
a generating means that generates a predetermined image on the basis of the index values; and
a display control means that identifiably displays, in displaying the predetermined image on a display means, a portion of pixels each having a mean of the luminance values, the mean being outside the predetermined range.

4. The information processing device according to claim 3, wherein the display control means displays, in displaying the predetermined image on the display means, the portion of the pixels each having the mean of the luminance values, the mean being outside the predetermined range, such that whether the mean is less than a lower limit value of the predetermined range or whether the mean is larger than an upper limit value of the predetermined range is able to be identified.

5. The information processing device according to claim 3, wherein
in generating the predetermined image on the basis of the index values, the generating means generates the predetermined image such that a predetermined color of the each pixel has lightness, hue, or chroma corresponding to a magnitude of the index value, and
in displaying the predetermined image on the display means, the display control means identifiably displays the portion of the pixels each having the mean of the luminance values, the mean being outside the predetermined range, by displaying the portion in a color other than the predetermined color.

6. The information processing device according to claim 3, wherein an upper limit value of the predetermined range is set on the basis of a gradation number of luminance in the speckle image.

7. The information processing device according to claim 3, wherein a lower limit value of the predetermined range is set on the basis of a standard deviation of noise in the speckle image.

8. An information processing method, including
an acquiring process of acquiring a speckle image from an imaging means that captures an image of reflected light of coherent light with which a subject is irradiated;
a calculating process of performing, for each pixel of the speckle image, on the basis of luminance values of that pixel and surrounding pixels, statistical processing and calculation of a predetermined index value;
a determining process of determining, for the each pixel, whether or not a mean of the luminance values used in the calculation of the index value is in a predetermined range;
a generating process of generating a predetermined image on the basis of the index values; and
a display control process of identifiably displaying, in displaying the predetermined image on a display means, a portion of pixels each having a mean of the luminance values, the mean being outside the predetermined range.
